(19) United States
(12) Patent Application Publication
(10) Pub. No.: US 2007/0134692 A1
(43) Pub. Date: Jun. 14, 2007

(54) METHOD, SYSTEM AND, COMPUTER SOFTWARE FOR EFFICIENT UPDATE OF PROBE ARRAY ANNOTATION DATA

(75) Inventors: Venu Valmeekam, El Cerrito, CA (US); Stephen Chervitz Trutane, Albany, CA (US); Adam N. Tracy, Berkeley, CA (US); Ronald T. Shigeta, Berkeley, CA (US); Benjamin Lai, Mountain View, CA (US)

Correspondence Address: AFFYMETRIX, INC;ATTN: CHIEF IP COUNSEL, LEGAL DEPT., 3420 CENTRAL EXPRESSWAY, SANTA CLARA, CA, 95051, US

(73) Assignee: Affymetrix, INC., Santa Clara, CA (US)

(21) Appl. No.: 11/567,685

(22) Filed: Dec. 6, 2006

Related U.S. Application Data
(60) Provisional application No. 60/748,884, filed on Dec. 9, 2005.
(60) Provisional application No. 60/759,090, filed on Jan. 13, 2006.

(51) Int. Cl. C12Q 1/68; G06F 19/00

(52) U.S. Cl. 435/6; 702/20

(57) ABSTRACT

In one embodiment, a method of assigning transcripts to probe sets is described that comprises retrieving one or more data sets from one or more sources; generating one or more clusters using transcript data from the data sets; aligning one or more probe sequences to a representative sequence from one or more of the clusters; aligning the representative sequence to a genome sequence, wherein the genome sequence is annotated with probe location information; mapping the aligned probe sequences to the genome sequence using the alignment of the representative sequence and genome sequence; and computing a score using a number associated with the aligned probe sequences and a number associated with the probe location formation associated with a region of the genome sequence that corresponds to the aligned representative sequence.

The present application claims priority to U.S. Provisional Applications 60/748,884 filed on Dec. 9, 2005 and 60/759,090 filed on Jan. 13, 2006, the disclosure of which are incorporated herein by reference in their entireties for all purposes.

### BACKGROUND

1. Field of the Invention

The present invention relates to the field of bioinformatics. In particular, the present invention relates to computer systems, methods, and products for analyzing genomic information and providing genomic information over networks such as the Internet. In particular the present invention relates to updating probe array annotation data at regular intervals using a method that provides a measure of reliability with respect to assignment of annotation information, where the annotations may subsequently be provided to a user in response to the user's request.

2. Related Art

Research in molecular biology, biochemistry, and many related health fields increasingly requires organization and analysis of complex data generated by new experimental techniques. These tasks are addressed by the rapidly evolving field of bioinformatics. See, e.g., H. Rashidi and K. Buehler, Bioinformatics Basics: Applications in Biological Science and Medicine (CRC Press, London, 2000); Bioinformatics: A Practical Guide to the Analysis of Gene and Proteins (B. F. Ouelette and A. D. Baxevanis, eds., Wiley & Sons, Inc.; 2d ed., 2001), both of which are hereby incorporated herein by reference in their entireties. Broadly, one area of bioinformatics applies computational techniques to large genomic databases, often distributed over and accessed through networks such as the Internet, for the purpose of illuminating relationships among alternative splice variants, protein function, and metabolic processes.

### SUMMARY OF THE INVENTION

Systems, methods, and products to address these and other needs are described herein with respect to illustrative, non-limiting, implementations. Various alternatives, modifications and equivalents are possible. For example, certain systems, methods, and computer software products are described herein using exemplary implementations for analyzing data from arrays of biological materials produced by the Affymetrix® 417™ or 427™ Arrayer. Other illustrative implementations are referred to in relation to data from Affymetrix® GeneChip® probe arrays. However, these systems, methods, and products may be applied with respect to many other types of probe arrays and, more generally, with respect to numerous parallel biological assays produced in accordance with other conventional technologies and/or produced in accordance with techniques that may be developed in the future. For example, the systems, methods, and products described herein may be applied to parallel assays of nucleic acids, PCR products generated from cDNA clones, proteins, antibodies, or many other biological materials. These materials may be disposed on slides (as typically used for spotted arrays), on substrates employed for GeneChip® arrays, or on beads, optical fibers, or other substrates or media, which may include polymeric coatings or other layers on top of slides or other substrates. Moreover, the probes need not be immobilized in or on a substrate, and, if immobilized, need not be disposed in regular patterns or arrays. For convenience, the term “probe array” will generally be used broadly hereafter to refer to all of these types of arrays and parallel biological assays.

In one embodiment, a method of assigning transcripts to probe sets is described that comprises retrieving one or more data sets from one or more sources; generating one or more clusters using transcript data from the data sets; aligning one or more probe sequences to a representative sequence from one or more of the clusters; aligning the representative sequence to a genome sequence, wherein the genome sequence is annotated with probe location information; mapping the aligned probe sequences to the genome sequence using the alignment of the representative sequence and genome sequence; and computing a score using a number associated with the aligned probe sequences and a number associated with the probe location formation associated with a region of the genome sequence that corresponds to the aligned representative sequence.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures or method steps and the leftmost digit of a reference numeral indicates the number of the figure in which the referenced element first appears (for example, the element 160 appears first in FIG. 1). In functional block diagrams, rectangles generally indicate functional elements and parallelograms generally indicate data. In method flow charts, rectangles generally indicate method steps and diamond shapes generally indicate decision elements. All of these conventions, however, are intended to be typical or illustrative, rather than limiting.

FIG. 1 is a functional block diagram of one embodiment of a web server in communication with remote databases and a computer over a network;

FIG. 2 is a functional block diagram of one embodiment of the computer of FIG. 1 comprising display devices that displays one or more web pages communicated from the web server over the network;

FIG. 3 is a functional block diagram of one embodiment of the web server and remote databases of FIG. 1, wherein the web server comprises an input manager, an output manager, and a database manager;

FIG. 4 is a functional block diagram of one embodiment of a first method for updating probe set annotation information using one or more data sets from one or more of the remote databases of FIGS. 1 and 3;

FIG. 5 is a functional block diagram of one embodiment of a second method for updating probe set annotation information using one or more data sets from one or more of the remote databases of FIGS. 1 and 3; and

FIG. 6 is a simplified graphical example of one embodiment of a graphical user interface for providing the updated annotation information generated by the methods of FIGS. 4 and 5 to a user.

### DETAILED DESCRIPTION

a) General

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W.H. Freeman Pub., New York, N.Y. and Berg et al. (2002) Biochemistry, 5th Ed., W.H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841; WO 00/58516; U.S. Pat. Nos. 5,143,854; 5,242,974; 5,252,743; 5,324,633; 5,384,261; 5,405,783; 5,424,186; 5,451,683; 5,482,867; 5,491,074; 5,527,681; 5,550,215; 5,571,639; 5,578,832; 5,593,839; 5,599,695; 5,624,711; 5,631,734; 5,795,716; 5,831,070; 5,837,832; 5,856,101; 5,858,659; 5,936,324; 5,968,740; 5,974,164; 5,981,185; 5,981,956; 6,025,601; 6,033,860; 6,040,193; 6,090,555; 6,136,269; 6,269,846; and 6,428,752; in PCT Applications Nos. PCT/US99/00730 (International Publication No. WO 99/36760); and PCT/US01/04285 (International Publication No. WO 01/58593); which are all incorporated herein by reference in their entirety for all purposes.

Patents that describe synthesis techniques in specific embodiments include U.S. Pat. Nos. 5,412,087; 6,147,205; 6,262,216; 6,310,189; 5,889,165; and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992; 6,013,449; 6,020,135; 6,033,860; 6,040,138; 6,177,248; and 6,309,822. Genotyping and uses therefore are shown in U.S. Ser. Nos. 10/442,021; 10/013,598 (U.S. Patent Application Publication 20030036069); and U.S. Pat. Nos. 5,856,092; 6,300,063; 5,858,659; 6,284,460; 6,361,947; 6,368,799; and 6,333,179. Other uses are embodied in U.S. Pat. Nos. 5,871,928; 5,902,723; 6,045,996; 5,541,061; and 6,197,506.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202; 4,683,195; 4,800,159; 4,965,188; and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5, 413,909; 5,861,245) and nucleic acid based sequence amplification (NABSA). (See, U.S. Pat. Nos. 5,409,818; 5,554,517; and 6,063,603, each of which is incorporated herein by reference). Other amplification methods that may be used are described in, U.S. Pat. Nos. 5,242,794; 5,494,810; 4,988,617; and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. No. 6,361,947, 6,391,592 and U.S. Ser. Nos. 09/916,135; 09/920,491 (U.S. Patent Application Publication 20030096235); Ser. No. 09/910,292 (U.S. Patent Application Publication 20030082543); and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y, 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928; 5,874,219; 6,045,996; 6,386,749; and 6,391,623 each of which are incorporated herein by reference.

The present invention also contemplates signal detection of hybridization between ligands in certain preferred embodiments. For example, methods and apparatus for signal detection and processing of intensity data are disclosed in, U.S. Pat. Nos. 5,143,854; 5,547,839; 5,578,832; 5,631,734; 5,800,992; 5,834,758; 5,856,092; 5,902,723; 5,936,324; 5,981,956; 6,025,601; 6,090,555; 6,141,096; 6,171,793; 6,185,030; 6,201,639; 6,207,960; 6,218,803; 6,225,625; 6,252,236; 6,335,824; 6,403,320; 6,407,858; 6,472,671; 6,490,533; 6,650,411; and 6,643,015, in U.S. patent application Ser. Nos. 10/389,194; 60/493,495; and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,733,729; 5,593,839; 5,795,716; 5,733,729; 5,974,164; 6,066,454; 6,090,555; 6,185,561; 6,188,783; 6,223,127; 6,228,593; 6,229,911; 6,242,180; 6,308,170; 6,361,937; 6,420,108; 6,484,183; 6,505,125; 6,510,391; 6,532,462; 6,546,340; and 6,687,692.

Additionally, the present invention may have preferred embodiments that include methods for providing genetic information over networks such as the Internet as shown in U.S. Ser. Nos. 10/197,621; 10/063,559 (United States Publication Number 20020183936); 10/065,856; 10/065,868; 10/328,818; 10/328,872; 10/423,403; and 60/482,389.

b) Definitions

The term “admixture” refers to the phenomenon of gene flow between populations resulting from migration. Admixture can create linkage disequilibrium (LD).

The term “allele” as used herein is any one of a number of alternative forms a given locus (position) on a chromosome. An allele may be used to indicate one form of a polymorphism, for example, a biallelic SNP may have possible alleles A and B. An allele may also be used to indicate a particular combination of alleles of two or more SNPs in a given gene or chromosomal segment. The frequency of an allele in a population is the number of times that specific allele appears divided by the total number of alleles of that locus.

The term “array” as used herein refers to an intentionally created collection of molecules which can be prepared either synthetically or biosynthetically. The molecules in the array can be identical or different from each other. The array can assume a variety of formats, for example, libraries of soluble molecules; libraries of compounds tethered to resin beads, silica chips, or other solid supports.

The term “biomonomer” as used herein refers to a single unit of biopolymer, which can be linked with the same or other biomonomers to form a biopolymer (for example, a single amino acid or nucleotide with two linking groups one or both of which may have removable protecting groups) or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

The term “biopolymer” or sometimes refer by “biological polymer” as used herein is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

The term “biopolymer synthesis” as used herein is intended to encompass the synthetic production, both organic and inorganic, of a biopolymer. Related to a bioploymer is a “biomonomer”.

The term “combinatorial synthesis strategy” as used herein refers to a combinatorial synthesis strategy is an ordered strategy for parallel synthesis of diverse polymer sequences by sequential addition of reagents which may be represented by a reactant matrix and a switch matrix, the product of which is a product matrix. A reactant matrix is a l column by m row matrix of the building blocks to be added. The switch matrix is all or a subset of the binary numbers, preferably ordered, between l and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

The term “complementary” as used herein refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “effective amount” as used herein refers to an amount sufficient to induce a desired result.

The term “genome” as used herein is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

The term “genotype” as used herein refers to the genetic information an individual carries at one or more positions in the genome. A genotype may refer to the information present at a single polymorphism, for example, a single SNP. For example, if a SNP is biallelic and can be either an A or a C then if an individual is homozygous for A at that position the genotype of the SNP is homozygous A or AA. Genotype may also refer to the information present at a plurality of polymorphic positions.

The term “Hardy-Weinberg equilibrium” (HWE) as used herein refers to the principle that an allele that when homozygous leads to a disorder that prevents the individual from reproducing does not disappear from the population but remains present in a population in the undetectable heterozygous state at a constant allele frequency.

The term “hybridization” as used herein refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.” Hybridizations are usually performed under stringent conditions, for example, at a salt concentration of no more than about 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations or conditions of 100 mM MES, 1 M [Na+], 20 mM EDTA, 0.01% Tween-20 and a temperature of 30-50° C., preferably at about 45-50° C. Hybridizations may be performed in the presence of agents such as herring sperm DNA at about 0.1 mg/ml, acetylated BSA at about 0.5 mg/ml. As other factors may affect the stringency of hybridization, including base composition and length of the complementary strands, presence of organic solvents and extent of base mismatching, the combination of parameters is more important than the absolute measure of any one alone. Hybridization conditions suitable for microarrays are described in the Gene Expression Technical Manual, 2004 and the GeneChip Mapping Assay Manual, 2004.

The term “hybridization probes” as used herein are oligonucleotides capable of binding in a base-specific manner to a complementary strand of nucleic acid. Such probes include peptide nucleic acids, as described in Nielsen et al., Science 254, 1497-1500 (1991), LNAs, as described in Koshkin et al. Tetrahedron 54:3607-3630, 1998, and U.S. Pat. No. 6,268,490, aptamers, and other nucleic acid analogs and nucleic acid mimetics.

The term “hybridizing specifically to” as used herein refers to the binding, duplexing, or hybridizing of a molecule only to a particular nucleotide sequence or sequences under stringent conditions when that sequence is present in a complex mixture (for example, total cellular) DNA or RNA.

The term “initiation biomonomer” or “initiator biomonomer” as used herein is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

The term “isolated nucleic acid” as used herein mean an object species invention that is the predominant species present (i.e., on a molar basis it is more abundant than any other individual species in the composition). Preferably, an isolated nucleic acid comprises at least about 50, 80 or 90% (on a molar basis) of all macromolecular species present. Most preferably, the object species is purified to essential homogeneity (contaminant species cannot be detected in the composition by conventional detection methods).

The term “ligand” as used herein refers to a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (for example, opiates, steroids, etc.), hormone receptors, peptides, enzymes, enzyme substrates, substrate analogs, transition state analogs, cofactors, drugs, proteins, and antibodies.

The term “linkage analysis” as used herein refers to a method of genetic analysis in which data are collected from affected families, and regions of the genome are identified that co-segregated with the disease in many independent families or over many generations of an extended pedigree. A disease locus may be identified because it lies in a region of the genome that is shared by all affected members of a pedigree.

The term “linkage disequilibrium” or sometimes referred to as “allelic association” as used herein refers to the preferential association of a particular allele or genetic marker with a specific allele, or genetic marker at a nearby chromosomal location more frequently than expected by chance for any particular allele frequency in the population. For example, if locus X has alleles A and B, which occur equally frequently, and linked locus Y has alleles C and D, which occur equally frequently, one would expect the combination AC to occur with a frequency of 0.25. If AC occurs more frequently, then alleles A and C are in linkage disequilibrium. Linkage disequilibrium may result from natural selection of certain combination of alleles or because an allele has been introduced into a population too recently to have reached equilibrium with linked alleles. The genetic interval around a disease locus may be narrowed by detecting disequilibrium between nearby markers and the disease locus. For additional information on linkage disequilibrium see Ardlie et al., Nat. Rev. Gen. 3:299-309, 2002.

The term “mendelian inheritance” as used herein refers to The term “lod score” or “LOD” is the log of the odds ratio of the probability of the data occurring under the specific hypothesis relative to the null hypothesis. LOD=log [probability assuming linkage/probability assuming no linkage].

The term “mixed population” or sometimes refer by “complex population” as used herein refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

The term “monomer” as used herein refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

The term “mRNA” or sometimes refer by “mRNA transcripts” as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

The term “nucleic acid library” or sometimes refer by “array” as used herein refers to an intentionally created collection of nucleic acids which can be prepared either synthetically or biosynthetically and screened for biological activity in a variety of different formats (for example, libraries of soluble molecules; and libraries of oligos tethered to resin beads, silica chips, or other solid supports). Additionally, the term “array” is meant to include those libraries of nucleic acids which can be prepared by spotting nucleic acids of essentially any length (for example, from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

The term “nucleic acids” as used herein may include any polymer or oligomer of pyrimidine and purine bases, preferably cytosine, thymine, and uracil, and adenine and guanine, respectively. See Albert L. Lehninger, Principles of Biochemistry, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

The term “oligonucleotide” or sometimes refer by “polynucleotide” as used herein refers to a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

The term “polymorphism” as used herein refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

The term “primer” as used herein refers to a single-stranded oligonucleotide capable of acting as a point of initiation for template-directed DNA synthesis under suitable conditions for example, buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

The term “probe” as used herein refers to a surface-immobilized molecule that can be recognized by a particular target. See U.S. Pat. No. 6,582,908 for an example of arrays having all possible combinations of probes with 10, 12, and more bases. Examples of probes that can be investigated by this invention include, but are not restricted to, agonists and antagonists for cell membrane receptors, toxins and venoms, viral epitopes, hormones (for example, opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

The term “receptor” as used herein refers to a molecule that has an affinity for a given ligand. Receptors may be naturally-occurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

The term “solid support”, “support”, and “substrate” as used herein are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

The term “target” as used herein refers to a molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

### c) EMBODIMENTS OF THE PRESENT INVENTION

Embodiments of a method for updating annotation information related to probes disposed upon a biological probe array are described herein that provide a user with a high degree of reliability with respect to assignment of information such as for instance transcript information with corresponding probes. In particular, embodiments are described that assign a grade to each assignment that are dependent upon a measure of the quality of the assignment.

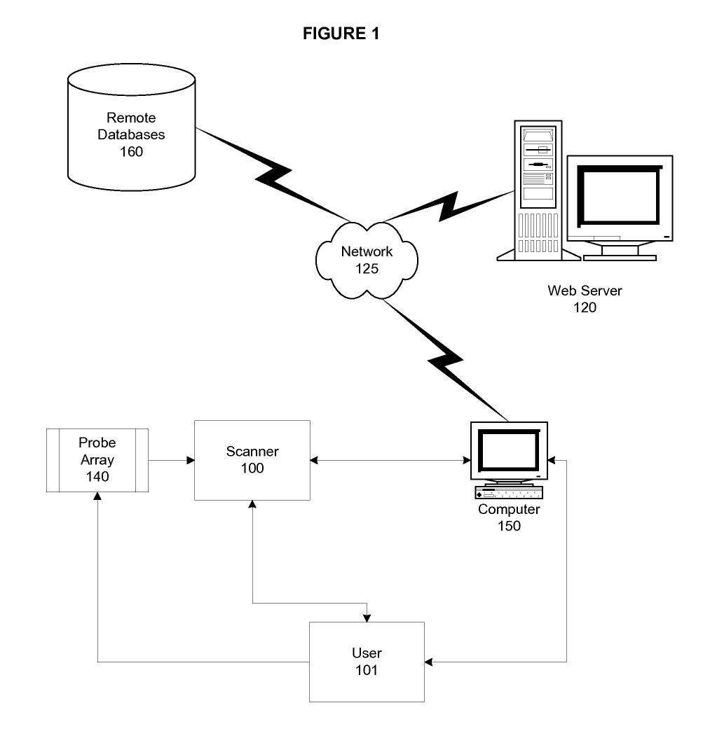

Probe Array 140: An illustrative example of probe array 140 is provided in FIG. 1. Descriptions of probe arrays are provided above with respect to “Nucleic Acid Probe arrays” and other related disclosure. In various implementations, probe array 140 may be disposed in a cartridge or housing such as, for example, the GeneChip® probe array available from Affymetrix, Inc. of Santa Clara Calif. Examples of probe arrays and associated cartridges or housings may be found in U.S. Pat. Nos. 5,945,334, 6,287,850, 6,399,365, 6,551,817, each of which is also hereby incorporated by reference herein in its entirety for all purposes. In addition, some embodiments of probe array 140 may be associated with pegs or posts, where for instance probe array 140 may be affixed via gluing, welding, or other means known in the related art to the peg or post that may be operatively coupled to a tray, strip or other type of similar substrate. Examples with embodiments of probe array 140 associated with pegs or posts may be found in U.S. patent Ser. No. 10/826,577, titled “Immersion Array Plates for Interchangeable Microtiter Well Plates”, filed Apr. 16, 2004, which is hereby incorporated by reference herein in its entirety for all purposes.

Scanner 100: Labeled targets hybridized to probe arrays may be detected using various devices, sometimes referred to as scanners, as described above with respect to methods and apparatus for signal detection. An illustrative device is shown in FIG. 1 as scanner 100. For example, scanners image the targets by detecting fluorescent or other emissions from labels associated with target molecules, or by detecting transmitted, reflected, or scattered radiation. A typical scheme employs optical and other elements to provide excitation light and to selectively collect the emissions.

For example, scanner 100 provides a signal representing the intensities (and possibly other characteristics, such as color that may be associated with a detected wavelength) of the detected emissions or reflected wavelengths of light, as well as the locations on the substrate where the emissions or reflected wavelengths were detected. Typically, the signal includes intensity information corresponding to elemental sub-areas of the scanned substrate. The term “elemental” in this context means that the intensities, and/or other characteristics, of the emissions or reflected wavelengths from this area each are represented by a single value. When displayed as an image for viewing or processing, elemental picture elements, or pixels, often represent this information. Thus, in the present example, a pixel may have a single value representing the intensity of the elemental sub-area of the substrate from which the emissions or reflected wavelengths were scanned. The pixel may also have another value representing another characteristic, such as color, positive or negative image, or other type of image representation. The size of a pixel may vary in different embodiments and could include a 2.5 μm, 1.5 μm, 1.0 μm, or sub-micron pixel size. Two examples where the signal may be incorporated into data are data files in the form *.dat or *.tif as generated respectively by Affymetrix® Microarray Suite (described in U.S. patent application Ser. No. 10/219,882, which is hereby incorporated by reference herein in its entirety for all purposes) or Affymetrix® GeneChip® Operating Software (described in U.S. patent application Ser. No. 10/764,663, which is hereby incorporated by reference herein in its entirety for all purposes) based on images scanned from GeneChip® arrays, and Affymetrix® Jaguar™ software (described in U.S. patent application Ser. No. 09/682,071, which is hereby incorporated by reference herein in its entirety for all purposes) based on images scanned from spotted arrays. Examples of scanner systems that may be implemented with embodiments of the present invention include U.S. patent application Ser. Nos. 10/389,194; and 10/913,102, both of which are incorporated by reference above; and U.S. patent application Ser. No. 10/846,261, titled “System, Method, and Product for Providing A Wavelength-Tunable Excitation Beam”, filed May 13, 2004; and U.S. patent application Ser. No. 11/260,617, titled “System, Method and Product for Multiple Wavelength Detection Using Single Source Excitation”, filed Oct. 27, 2005, each of which is hereby incorporated by reference herein in its entirety for all purposes.

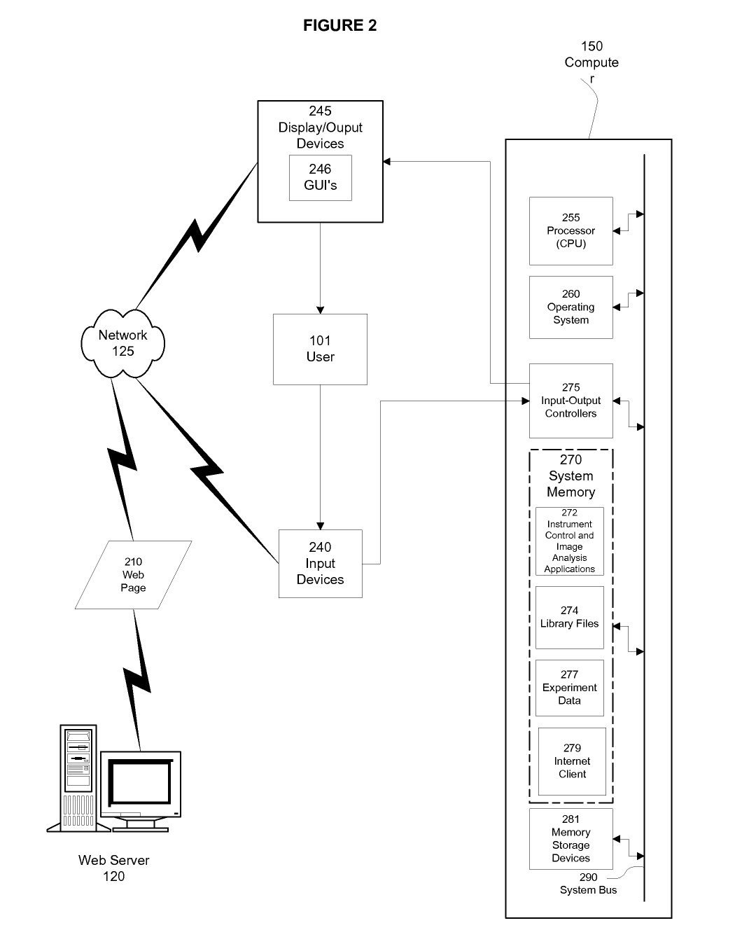

Computer 150: An illustrative example of computer 150 is provided in FIG. 1 and also in greater detail in FIG. 2. Computer 150 may be any type of computer platform such as a workstation, a personal computer, a server, or any other present or future computer. Computer 150 typically includes known components such as a processor 255, an operating system 260, system memory 270, memory storage devices 281, and input-output controllers 275, input devices 240, and display/output devices 245. Display/Output Devices 245 may include display devices that provides visual information, this information typically may be logically and/or physically organized as an array of pixels. A Graphical user interface (GUI) controller may also be included that may comprise any of a variety of known or future software programs for providing graphical input and output interfaces such as for instance GUI's 246. For example, GUI's 246 may provide one or more graphical representations on one or more display devices to a user, such as user 101, and also be enabled to process user inputs via GUI's 246 using means of selection or input known to those of ordinary skill in the related art.

It will be understood by those of ordinary skill in the relevant art that there are many possible configurations of the components of computer 150 and that some components that may typically be included in computer 150 are not shown, such as cache memory, a data backup unit, and many other devices. Processor 255 may be a commercially available processor such as an Itanium® or Pentium® processor made by Intel Corporation, a SPARC® processor made by Sun Microsystems, an Athalon™ or Opteron™ processor made by AMD corporation, or it may be one of other processors that are or will become available. Processor 255 executes operating system 260, which may be, for example, a Windows®-type operating system (such as Windows NT® 4.0 with SP6a, or Windows XP) from the Microsoft Corporation; a Unix® or Linux-type operating system available from many vendors or what is referred to as an open source; another or a future operating system; or some combination thereof. Operating system 260 interfaces with firmware and hardware in a well-known manner, and facilitates processor 255 in coordinating and executing the functions of various computer programs that may be written in a variety of programming languages. Operating system 260, typically in cooperation with processor 255, coordinates and executes functions of the other components of computer 150. Operating system 260 also provides scheduling, input-output control, file and data management, memory management, and communication control and related services, all in accordance with known techniques.

System memory 270 may be any of a variety of known or future memory storage devices. Examples include any commonly available random access memory (RAM), magnetic medium such as a resident hard disk or tape, an optical medium such as a read and write compact disc, or other memory storage device. Memory storage devices 281 may be any of a variety of known or future devices, including a compact disk drive, a tape drive, a removable hard disk drive, flash memory, or a diskette drive. Such types of memory storage devices 281 typically read from, and/or write to, a program storage medium (not shown) such as, respectively, a compact disk, magnetic tape, removable hard disk, flash memory, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory 270 and/or the program storage device used in conjunction with memory storage device 281.

In some embodiments, a computer program product is described comprising a computer usable medium having control logic (computer software program, including program code) stored therein. The control logic, when executed by processor 255, causes processor 255 to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers 275 could include any of a variety of known devices for accepting and processing information from a user, whether a human or a machine, whether local or remote. Such devices include, for example, modem cards, network interface cards, sound cards, or other types of controllers for any of a variety of known input devices. Output controllers of input-output controllers 275 could include controllers for any of a variety of known display devices for presenting information to a user, whether a human or a machine, whether local or remote. In the illustrated embodiment, the functional elements of computer 150 communicate with each other via system bus 290. Some of these communications may be accomplished in alternative embodiments using network or other types of remote communications.

As will be evident to those skilled in the relevant art, instrument control and image processing applications 272, if implemented in software, may be loaded into and executed from system memory 270 and/or memory storage device 281. All or portions of applications 272 may also reside in a read-only memory or similar device of memory storage device 281, such devices not requiring that applications 272 first be loaded through input-output controllers 275. It will be understood by those skilled in the relevant art that applications 272, or portions of it, may be loaded by processor 255 in a known manner into system memory 270, or cache memory (not shown), or both, as advantageous for execution. Also illustrated in FIG. 2 are library files 274, experiment data 277, and internet client 279 stored in system memory 270. For example, experiment data 277 could include data related to one or more experiments or assays such as excitation wavelength ranges, emission wavelength ranges, extinction coefficients and/or associated excitation power level values, or other values associated with one or more fluorescent labels. Additionally, internet client 279 may include an application enabled to accesses a remote service on another computer using a network that may for instance comprise what are generally referred to as “Web Browsers”. In the present example some commonly employed web browsers include Netscape® 8.0 available from Netscape Communications Corp., Microsoft® Internet Explorer 6 with SP1 available from Microsoft Corporation, Mozilla Firefox® 1.5 from the Mozilla Corporation, or other type of web browser currently known in the art or to be developed in the future. Also, in the same or other embodiments internet client 279 may include, or could be an element of, specialized software applications enabled to access remote information via a network such as network 125 such as, for instance, the GeneChip® Data Analysis Software (GDAS) package or Chromosome Copy Number Tool (CNAT) both available from Affymetrix, Inc. of Santa Clara Calif. that are each enabled to access information, and in particular probe array annotation information from the NetAffx™ web site hosted on one or more servers provided by Affymetrix, Inc.

Network 125 may include one or more of the many various types of networks well known to those of ordinary skill in the art. For example, network 125 may include a local or wide area network that employs what is commonly referred to as a TCP/IP protocol suite to communicate, that may include a network comprising a worldwide system of interconnected computer networks that is commonly referred to as the internet, or could also include various intranet architectures.

Instrument control and image processing applications 272: Instrument control and image processing applications 272 may be any of a variety of known or future image processing applications. Examples of applications 272 include Affymetrix® Microarray Suite, Affymetrix® GeneChip® Operating Software (hereafter to as GCOS), and Affymetrix® Jaguar™ software, noted above. Applications 272 may be loaded into system memory 270 and/or memory storage device 281 through one of input devices 240.

Embodiments of applications 272 include executable code being stored in system memory 270 of an implementation of computer 150. Applications 272 may provide a user interface for both the client workstation and one or more servers such as, for instance, GeneChip® Operating Software Server (GCOS Server) available from Affymetrix, Inc. Santa Clara, Calif. Applications 272 could additionally provide the user interface for one or more other workstations and/or one or more instruments. In the presently described implementation, the interface may communicate with and control one or more elements of the one or more servers, one or more workstations, and the one or more instruments. In the described implementation the client workstation could be located locally or remotely to the one or more servers and/or one or more other workstations, and/or one or more instruments. The user interface may, in the present implementation, include an interactive graphical user interface (generally referred to as a GUI), such as GUI's 246, that allow a user to make selections based upon information presented in the GUI. For example, applications 272 may provide GUI 246 that allows a user to select from a variety of options including data selection, experiment parameters, calibration values, probe array information. Applications 272 may also provide a graphical representation of raw or processed image data where the processed image data may also include annotation information superimposed upon the image such as, for instance, base calls, features of probe array 140, or other useful annotation information. Further examples of providing annotation information on image data are provided in U.S. Provisional Patent Application Ser. No. 60/493,950, titled “System, Method, and Product for Displaying Annotation Information Associated with Microarray Image Data”, filed Aug. 8, 2003, which is hereby incorporated by reference herein in its entirety for all purposes.

In alternative implementations, applications 272 may be executed on a server, or on one or more other computer platforms connected directly or indirectly (e.g., via another network, including the Internet or an Intranet) to network 125.

Embodiments of applications 272 also include instrument control features. The instrument control features may include the control of one or more elements of one or more instruments that could, for instance, include elements of a fluid processing station, what may be referred to as an automatic cartridge or tray loader, one or more robotic elements, and scanner 100. The instrument control features may also be capable of receiving information from the one more instruments that could include experiment or instrument status, process steps, or other relevant information. The instrument control features could, for example, be under the control of or an element of the user interface. In the present example, a user may input desired control commands and/or receive the instrument control information via one of GUI's 246. Additional examples of instrument control via a GUI or other interface is provided in U.S. Provisional Patent Application Ser. No. 60/483,812, titled “System, Method and Computer Software for Instrument Control, Data Acquisition and Analysis”, filed Jun. 30, 2003, which is hereby incorporated by reference herein in its entirety for all purposes.

In some embodiments, image data is operated upon by applications 272 to generate intermediate results. Examples of intermediate results include so-called cell intensity files (*.cel) and chip files (*.chp) generated by Affymetrix® GeneChip® Operating Software or Affymetrix® Microarray Suite (as described, for example, in U.S. patent application Ser. Nos. 10/219,882, and 10/764,663, both of which are hereby incorporated herein by reference in their entireties for all purposes) and spot files (*.spt) generated by Affymetrix® Jaguar™ software (as described, for example, in PCT Application PCT/US01/26390 and in U.S. patent applications Ser. Nos. 09/681,819, 09/682,071, 09/682,074, and 09/682,076, all of which are hereby incorporated by reference herein in their entireties for all purposes). For convenience, the term “file” often is used herein to refer to data generated or used by applications 272 and executable counterparts of other applications, but any of a variety of alternative techniques known in the relevant art for storing, conveying, and/or manipulating data may be employed.

For example, applications 272 receives image data derived from a GeneChip® probe array and generates a cell intensity file. This file contains, for each probe scanned by scanner 100, a single value representative of the intensities of pixels measured by scanner 100 for that probe. Thus, this value is a measure of the abundance of tagged mRNA's present in the target that hybridized to the corresponding probe. Many such mRNA's may be present in each probe, as a probe on a GeneChip® probe array may include, for example, millions of oligonucleotides designed to detect the mRNA's. As noted, another file illustratively assumed to be generated by applications 272 is a chip file. In the present example, in which applications 272 include Affymetrix® GeneChip® Operating Software, the chip file is derived from analysis of the *.cel file combined in some cases with information derived from lab data and/or library files 274 that specify details regarding the sequences and locations of probes and controls. The resulting data stored in the chip file includes degrees of hybridization, absolute and/or differential (over two or more experiments) expression, genotype comparisons, detection of polymorphisms and mutations, and other analytical results.

In another example, in which applications 272 includes Affymetrix® Jaguar™ software operating on image data from a spotted probe array, the resulting spot file includes the intensities of labeled targets that hybridized to probes in the array. Further details regarding cell files, chip files, and spot files are provided in U.S. patent application Ser. Nos. 09/682,074 incorporated by reference above, as well as 10/126,468; and 09/682,098; which are hereby incorporated by reference herein in their entireties for all purposes. As will be appreciated by those skilled in the relevant art, the preceding and following descriptions of files generated by applications 272 are exemplary only, and the data described, and other data, may be processed, combined, arranged, and/or presented in many other ways.

User 101 and/or automated data input devices or programs (not shown) may provide data related to the design or conduct of experiments. As one further non-limiting example related to the processing of an Affymetrix® GeneChip® probe array, the user may specify an Affymetrix catalogue or custom chip type (e.g., Human Genome U133 plus 2.0 chip) either by selecting from a predetermined list presented by GCOS or by scanning a bar code, Radio Frequency Identification (RFID), or other means of electronic identification related to a chip to read its type. GCOS may associate the chip type with various scanning parameters stored in data tables including the area of the chip that is to be scanned, the location of chrome borders on the chip used for auto-focusing, the wavelength or intensity/power of excitation light to be used in reading the chip, and so on. As noted, applications 285 may apply some of this data in the generation of intermediate results. For example, information about the dyes may be incorporated into determinations of relative expression.

Those of ordinary skill in the related art will appreciate that one or more operations of applications 272 may be performed by software or firmware associated with various instruments. For example, scanner 100 could include a computer that may include a firmware component that performs or controls one or more operations associated with scanner 100, such as for instance scanner computer 210 and scanner firmware 472.

Web Server 120: An example of web server 120 is illustrated in FIGS. 1 and 2 and may support one or more web sites or “portals” accessible remotely by computers, handheld devices, cell phones, or other web capable devices known in the art via network 125. Typical embodiments of server 120 may include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes, and other known devices or media and those that may be developed in the future. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Baxevanis Bioinformatics: A Practical Guide for Analysis of Gene and Proteins (Wiley & Sons, Inc., 2nd ed., 2001).

As will be appreciated by one of skill in the art, the present invention may be embodied as a method, data processing system or program products. Accordingly, the present invention may take the form of data analysis systems, methods, analysis software, and so on. Software written according to the present invention typically is to be stored in some form of computer readable medium, such as memory, or CD-ROM, or transmitted over a network, and executed by a processor. For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems : A Practical Guide for Systems Professionals by Paul E. Renaud, 2nd edition (June 1996), John Wiley & Sons; ISBN: 0471133337, both of which are hereby incorporated by reference for all purposes.

Computer software products may be written in any of various suitable programming languages, such as C, C++, Fortran and Java (Sun Microsystems). The computer software product may be an independent application with data input and data display modules. Alternatively, the computer software products may be classes that may be instantiated as distributed objects. The computer software products may also be component software such as Java Beans (Sun Microsystems), Enterprise Java Beans (EJB), Microsoft® COM/DCOM, etc.

Web Server 120 is illustrated in FIGS. 1 and 2 as a single computer platform or machine, but those of ordinary skill in the related art will appreciate that web server 120 may comprise one or more server platforms, and may typically comprise a plurality of server machines.

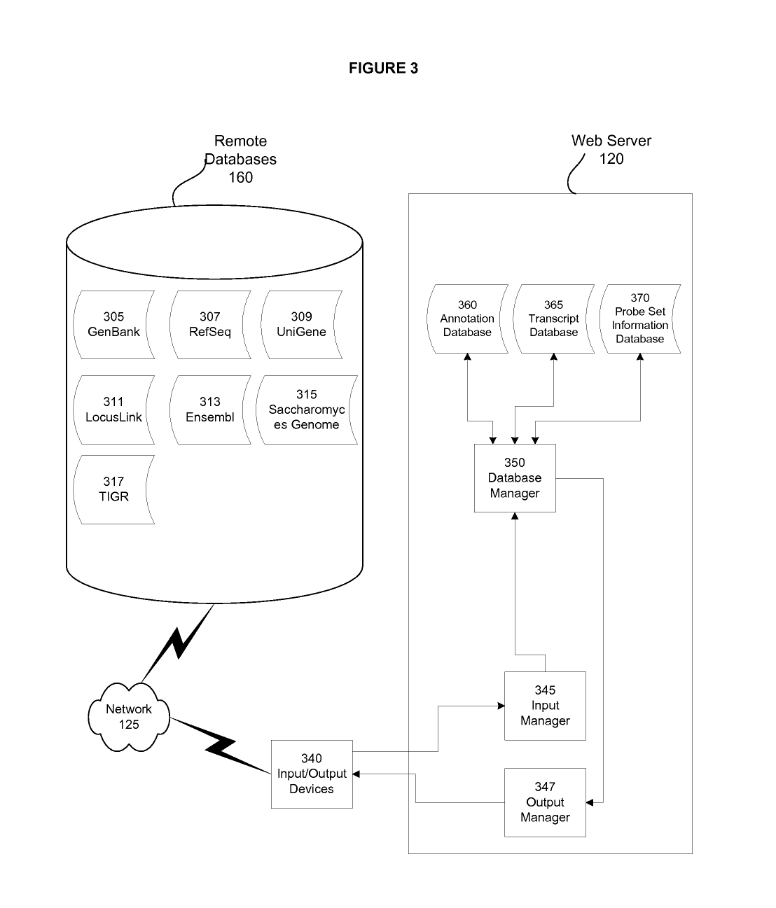

Illustrative examples of functional elements associated with server 120 as illustrated in FIG. 3, such as database manager 350, input and output managers 345 and 347. That is, in a typical implementation, the functions of managers 345 and 347 are carried out by the execution of software applications on and across one or more computer platforms represented by server 120.

For example, server 120 may be any type of known computer platform or a type to be developed in the future, although typically will be of a class of computer commonly referred to as servers. However, server 120 may also be a main frame computer, a work station, or other computer type. Server 120 may be connected via any known or future type of cabling or other communication system including wireless systems, either networked or otherwise. Various computing elements of server 120 may be co-located or they may be physically separated. Various operating systems may be employed on any of the computer platforms, possibly depending on the type and/or make of computer platform chosen. Appropriate operating systems include Windows NT®, Sun Solaris, Linux, OS/400, Compaq Tru64 Unix, SGI IRIX, Siemens Reliant Unix, and others.

There may be significant advantages to carrying out the functions of server 120 on multiple computer platforms, such as lower costs of deployment, database switching, or changes to enterprise applications, and/or more effective firewalls. Other configurations, however, are possible. For example, as is well known to those of ordinary skill in the relevant art, so-called two-tier or N-tier architectures are possible, ee, for example, E. Roman, Mastering Enterprise JavaBeans™ and the Java™2 Platform (Wiley & Sons, Inc., NY, 1999) and J. Schneider and R. Arora, Using Enterprise Java™ (Que Corporation, Indianapolis, 1997), both of which are hereby incorporated by reference in their entireties for all purposes.

It will be understood that many hardware and associated software or firmware components that may be implemented in a server-side architecture for Internet commerce may be included, as well as components to implement one or more firewalls to protect data and applications, uninterruptible power supplies, LAN switches, web-server routing software, and many other components. Similarly, a variety of computer components customarily included in server-class computing platforms, as well as other types of computers, will be understood to be included but are not shown. These components include, for example, processors, memory units, input/output devices, buses, and other components noted above with respect to user computer 100. Those of ordinary skill in the art will readily appreciate how these and other conventional components may be implemented.

The functional elements of server 120 also may be implemented in accordance with a variety of software facilitators and platforms (although it is not precluded that some or all of the functions of server 120 may also be implemented in hardware or firmware). Among the various commercial products available for implementing e-commerce web portals are BEA WebLogic from BEA Systems, which is a so-called “middleware” application. This and other middleware applications are sometimes referred to as “application servers”. The function of these middleware applications generally is to assist other software components to share resources and coordinate activities. The goals include making it easier to write, maintain, and change the software components; to avoid data bottlenecks; and prevent or recover from system failures. Thus, these middleware applications may provide load-balancing, fail-over, and fault tolerance, all of which features will be appreciated by those of ordinary skill in the relevant art.

Other development products, such as the Java™ 2 platform from Sun Microsystems, Inc. may be employed in server 120 to provide suites of applications programming interfaces (API's) that, among other things, enhance the implementation of scalable and secure components, examples of API's for use with probe array related information and architectures may be found in U.S. Pat. No. 6,954,699, which is hereby incorporated by reference herein in its entirety for all purposes. The platform known as J2EE (Java™2, Enterprise Edition), is configured for use with Enterprise JavaBeans™, both from Sun Microsystems. Enterprise JavaBeans™ generally facilitates the construction of server-side components using distributed object applications written in the Java™ language. Thus, in one implementation, the functional elements of server 120 may be written in Java and implemented using J2EE and Enterprise JavaBeans™. Various other software development approaches or architectures may be used to implement the functional elements of server 120 and their interconnection, as will be appreciated by those of ordinary skill in the art Some embodiments of web server 120 may perform methods for providing genetic information, and more specifically genetic information that pertains to biological probe arrays, over network 125 such as the Internet as described in U.S. patent application Ser. Nos. 10/063,559, 10/065,856; 10/065,868; 10/197,621; 10/328,872; 10/328,818; 10/423,403; 10/903,344; and 10/903,641, which are all hereby incorporated by reference herein in their entireties for all purposes.

For example, user 101 may provide one or more “probe-set identifiers” to server 120 via one or more of GUI's 246 for processing, where server 120 may return various types of information in response to particular request by user 101. Those of ordinary skill in the related art will appreciate that inputs to server 120 by user 101 are not necessarily limited to probe set identifiers, rather probe set identifiers provide a useful means to obtain information related to probes, probe arrays, and other associated information.

These probe-set identifiers typically come to the attention of user 101 as a result of experiments conducted on probe arrays. For example, user 101 may select probe-set identifiers that identify microarray probes or probe sets capable of enabling detection of the expression of mRNA transcripts from corresponding genes or ESTs of particular interest. As is well known in the relevant art, an EST is a fragment of a gene sequence that may not be fully characterized, whereas a gene sequence is generally understood to be complete and fully characterized. The term “gene” is used generally herein to refer both to full size genes of known sequence and to computationally predicted genes. In some implementations, the specific sequences detected by the arrays that represent these genes or ESTs may be referred to as, “sequence information fragments (SIF's)” and may be recorded in a “SIF file”. In particular implementations, a SIF is a portion of a consensus sequence that has been deemed to best represent the mRNA transcript from a given gene or EST. The consensus sequence may have been derived by comparing and clustering ESTs, and possibly also by comparing the ESTs to genomic sequence information. A SIF is a portion of the consensus sequence for which probes on the array are specifically designed. With respect to the operations of web server 120, it is assumed with respect to some implementations that some microarray probe sets may be designed to detect the expression of genes based upon sequences of ESTs.

As was described above, the term “probe set” refers in some implementations to one or more probes from an array of probes on a microarray. For example, in an Affymetrix® GeneChip® probe array, in which probes are synthesized on a substrate, a probe set may for instance comprise 9, 30, or 40 probe features depending on the design and application of probe array 140 for which they are designed, where each probe comprises a sequence that typically differs from that of the other probes in the probe set. These probes collectively, or in various combinations of some or all of them, are deemed to be indicative of a gene, EST, or protein. In a spotted probe array, one or more spots may similarly constitute a “probe set.” Similarly, each probe in a probe set may also comprise a unique identifier.

The term “probe-set identifiers” is used broadly herein in that a number of types of such identifiers are possible and are intended to be included within the meaning of this term. One type of probe-set identifier is a name, number, or other symbol for the purpose of identifying a probe set. This name, number, or symbol may be arbitrarily assigned to the probe set by, for example, the manufacturer of the probe array, user 101, or other who provides a definition of the probes and probe sets. A user may select this type of probe-set identifier by, for example, highlighting or typing the in one or more fields associated with GUI's 246. Another type of probe-set identifier as intended herein is a graphical representation of a probe set. For example, dots may be displayed on a scatter plot or other diagram wherein each dot represents a probe set. Typically, the dot's placement on the plot represents the intensity of the signal from hybridized, tagged, targets (as described in greater detail below) in one or more experiments. In these cases, a user may select a probe-set identifier by clicking on, drawing a loop around, or otherwise selecting one or more of the dots. In another example, user 101 may select a probe-set identifier by selecting a row or column in a table or spreadsheet that correlates probe sets with accession numbers and other genomic information.

Yet another type of probe-set identifier, as that term is used herein, includes a nucleotide or amino acid sequence. For example, it is illustratively assumed that a particular SIF is a unique sequence of 500 bases that is a portion of a consensus sequence or exemplar sequence gleaned from EST and/or genomic sequence information. It further is assumed that one or more probe sets are designed to represent the SIF. A user who specifies all or part of the 500-base sequence thus may be considered to have specified all or some of the corresponding probe sets.

A further example of a probe-set identifier is an accession number of a gene or EST. Gene and EST accession numbers are publicly available. A probe set may therefore be identified by the accession number or numbers of one or more ESTs and/or genes corresponding to the probe set. The correspondence between a probe set and ESTs or genes may be maintained in a suitable database from which the correspondence may be provided to user 101. Similarly, gene fragments or sequences other than ESTs may be mapped (e.g., by reference to a suitable database) to corresponding genes or ESTs for the purpose of using their publicly available accession numbers as probe-set identifiers. For example, a user may be interested in genomic information related to a particular SIF that is derived from EST-1 and EST-2. The user may be provided with the correspondence between that SIF (or part or all of the sequence of the SIF) and EST-1 or EST-2, or both. To obtain genomic data or analyze the sequence related to the SIF, or a partial sequence of it, the user may select the accession numbers of EST-1, EST-2, or both.

Additional examples of probe-set identifiers include one or more terms that may be associated with the annotation of one or more gene or EST sequences, where the gene or EST sequences may be associated with one or more probe sets. For convenience, such terms may hereafter be referred to as “annotation terms” and will be understood to potentially include, in various implementations, one or more words, graphical elements, characters, or other representational forms that provide information that typically is biologically relevant to or related to the gene or EST sequence. Examples of such terms associated with annotations include those of molecular function (e.g. transcription initiation), cellular location (e.g. nuclear membrane), biological process (e.g. immune response), tissue type (e.g. kidney), or other annotation terms known to those in the relevant art.

Typically, each gene or EST has at least one corresponding probe set that is identified by a probe-set identifier that, as just noted, may be a number, name, accession number, symbol, graphical representation (e.g., dot or highlighted tabular entry), and/or nucleotide sequence, as illustrative and non-limiting examples. The corresponding probe sets are capable of enabling detection of the expression of their corresponding gene or alternative splice variant. In some embodiments a probe set designed to recognize the mRNA expression of a gene may identify one or more alternative splice variants. In some cases a plurality of probe sets may be capable of identifying a specific alternative splice variant.

In response to a user selection of one or more probe-set identifiers, server 120 provides user 101 with annotation information that may include one or more of genomic, EST, protein, or other descriptive information. This information may be helpful to user 101 in analyzing the results of experiments and in designing or implementing follow-up experiments.

As noted, one of the functional elements of server 120 is input manager 345. Manager 345 receives a set, i.e., one or more, of probe-set identifiers from user 101 over network 125. These functions are performed in accordance with known techniques common to the operation of Internet servers, also commonly referred to in similar contexts as presentation servers. Another of the functional elements of server 120 is output manager 347. Manager 347 provides information assembled to user 101 over network 125, also in accordance with those known techniques. The presentation by manager 347 of data may be implemented in accordance with a variety of known techniques. As some examples, data may include HTML or XML documents, email or other files, or data in other forms. The data may include Internet URL addresses so that user 101 may retrieve additional HTML, XML, or other documents or data from remote sources.

Server 120 further includes database manager 350. In the illustrated embodiment, database manager 350 coordinates the storage, maintenance, supplementation, and all other transactions from or to any local databases. Manager 350 may undertake these functions in cooperation with appropriate database applications such as the Oracle®10 g database management system.

In some implementations, manager 350 periodically updates annotation database 360. The data updated in database 360 includes data related to genes, ESTs, or proteins that correspond with one or more probe sets. The probe sets may be those used or designed for use on any microarray product, and/or that are expected or calculated to be used in microarray products of any manufacturer or researcher. For example, the probe sets may include all probe sets synthesized on the line of stocked GeneChip® probe arrays from Affymetrix, Inc., including its Arabidopsis Genome Array, C. elegans Genome Array, Drosophila Genome Array, E. coli Genome Array, Human Genome Focus Array, Human Genome U133 Set, Human Genome U95 Set, Mouse Expression Set 430, Murine Genome U74v2 Set, P. aeruginosa Genome Array, Rat Expression Set 230, Rat Genome U34 Set, Rat Neurobiology U34 Array, Rat Toxicology U34 Array, Test3 Array, Yeast Genome S98 Array, CYP450 Array, GenFlex Tag Array, HuSNP Probe Array, 10K Probe Array, 100K Probe Array, 500K Probe Array, p53 Probe Array, Tiling Probe Array, and Exon Probe Array. The probe sets may also include those synthesized on alternative splice arrays or custom arrays for user 101 or others. However, the data updated in database 360 need not be so limited. Rather, it may relate, e.g., to any number of genes, ESTs, or proteins. Types of data that may be stored in database 360 are described below in relation to the operations of manager 350 in directing the periodic collection of this data from remote sources providing the locally maintained data in database 360 to users.

Database manager 350 may periodically update annotation database 360 from various sources, such as remote databases 160. For example, according to any chronological schedule (e.g., daily, weekly, etc.), or need-driven schedule (e.g., in response to a user making an authorized request for updated information), manager 350 may, in accordance with known techniques, initiate searches of remote databases 160 by formulating appropriate queries, addressed to the URL's of the various databases 160, or by other conventional techniques for conducting data searches and/or retrieving data or documents over the Internet. These search queries and corresponding addresses may be provided in a known manner to output manager 347 for presentation to databases 160. Input manager 345 receives replies to the queries and provides them to manager 350 for updating of database 360, all in accordance with any of a variety of known techniques for managing information flow to, from, and within an Internet site. Alternatively, in some embodiments updates to annotation database 360 may be performed manually by an operator employing one or more computers of server 120, where for instance the process of curating the data may require manual interventions by the operator, where the curated data may be subsequently uploaded to database 360.

An application manager may be employed to manage the administrative aspects of server 120, possibly with the assistance of a middleware product such as an applications server product. One of these administrative tasks may be the issuance of periodic instructions to manager 350 to initiate the periodic updating of database 360 just described. Alternatively, manager 350 may self-initiate this task. It is not required that all data in database 360 be updated according to the same periodic schedule. Rather, it may be typical for different types of data and/or data from different sources to be updated according to different schedules. Moreover, these schedules may be changed, and need not be according to a consistent schedule. That is, for example, updating for particular data may occur after a day, then again after 2 days, then at a different period that may continue to vary. Numerous factors may influence the determination by the application manager or manager 350 to maintain or vary these periods, such as the response time from various remote databases 160, the value and/or timeliness of the information in those databases, cost considerations related to accessing or licensing the databases, the quantity of information that must be accessed, and so on.

As described above with respect to probe set identifiers, many probe sets are originally designed using an SIF or consensus sequence derived from one or more EST fragments or a cluster of EST fragments, where the identity of a gene represented by those fragments and functional roles of the fragments are unknown. As those of ordinary skill in the related art will appreciate, the pace of scientific discovery with respect to identification of the gene identities and functional characteristics associated with probe sets has been rapid, and it is desirable in many applications to be able to provide a user such as user 101 with the most up to date and accurate information available that in turn increase the value derived from experiments using probe arrays 140. Therefore, preferred embodiments of the present invention include regular updates of annotation information, such as the described transcript assignment information, by for example database manager or in some alternative embodiments by manual curation or both as described above.

Some methods of annotating transcript assignment to probe sets have been employed that simply matched transcripts to entries in various databases such as LocusLink 311 or UniGene 309. It will be evident to those of ordinary skill in the related art that there is typically a level of redundancy of entries in various databases that are undesirable. In other words a transcript or fragments of transcripts associated with a gene may be represented multiple times in a single database and in some cases various entries may be associated with different information. In addition, there are instances where information between databases do not correlate with each other where for instance one dataset from a first database may classify a transcript as being associated with a particular gene or locus, and another dataset from a second different database may classify the same transcript as being associated with intronic regions of the locus that are defined as non-coding or a different locus.

Improved methods of annotating probe and probe set information of the presently described invention employ datasets from a plurality of available databases. For example, data manager 350 may perform one or more operations to calculate transcript assignments for probes and probe sets that comprise one or more steps to reduce redundancy and increase the accuracy of transcript representation. In the present example, data manager 350 may perform different methods of annotating probe and probe set information where the methods may be dependent upon the design of the probes. Preferred embodiments of the invention utilize datasets from what are generally referred to as “Non-Redundant” databases where attempts have already been made by the curators of each dataset to remove redundancy, although as those of ordinary skill in the art will appreciate some degree of redundancy will typically still exist. Still the employment of non-redundant datasets will generally improve the efficiency of the presently described inventions.

The presently described inventions are directed to methods, systems for performing the methods, and computer software that provides more reliable annotated assignments of transcripts to probes and probe sets and a measure of the quality of the assignment where the measure may be qualitative or quantitative. The utility of updating annotation information includes providing user 101 with the most accurate probe set annotation information currently available. For example it is advantageous for a user to employ the most up to date and accurate information available in regard to transcripts of interest that a probe or probe set interrogates because the biological interpretation generated from such improved information will be similarly improved. In the present example, user 101 may receive annotation information from server 120 in response to selecting one or more probe or probe set identifiers corresponding to probes or probe sets of interest using one or more GUI's 246.

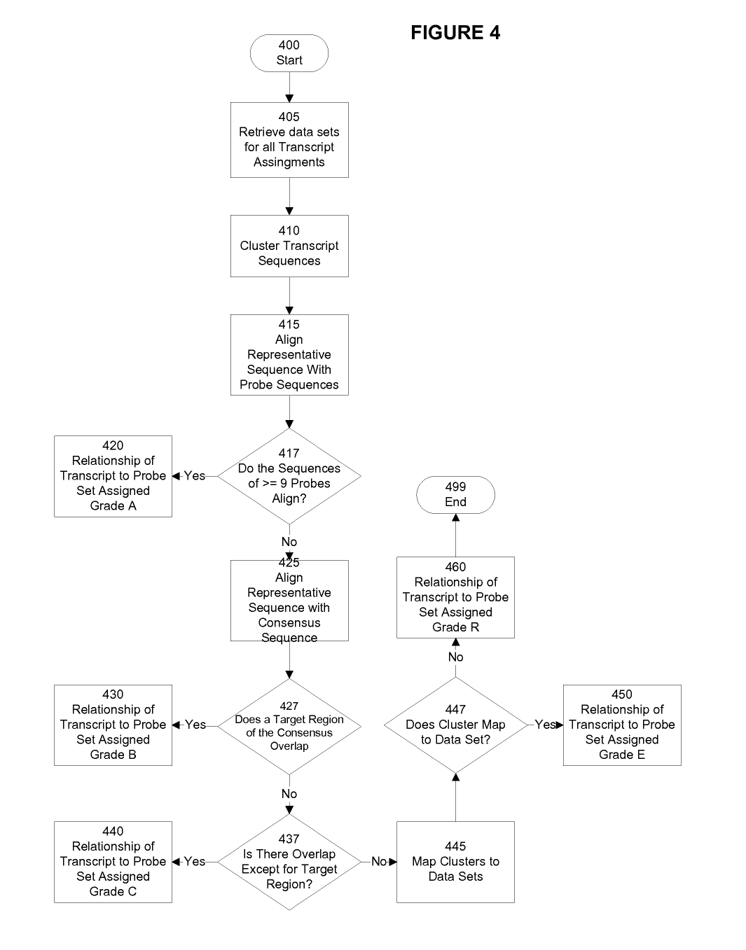

FIG. 4 provides an illustrative example of one embodiment of a method for updating annotation information associated with probe sets that interrogate the expression of transcripts from genes comprising one or more probes that target the 3′ end of the sequence that is representative of the gene of interest.

As illustrated in step 405, data sets for all transcripts or fragments of transcripts are downloaded from remote databases 160 via input manager 345 and input output devices 340. Those of ordinary skill in the related art will appreciate that the term “transcript” generally refers to products “transcribed” from a DNA template and may typically be referred to as mRNA, EST's, primary transcripts, or other terms generally used in the art. In some implementations manager 350 may store raw transcript data sets downloaded from one or more of databases 160 in transcript database 365. As stated above, it may be preferable in many implementations to employ non-redundant data sets offered by particular databases that may for instance include GenBank 305, RefSeq 307, UniGene 309, LocuLink 311, Ensembl 313, Saccharomyces Genome 315, TIGR 317, or any other database currently known in the related art or that may be developed in the future.

Next, step 410 illustrates the step where database manager 350 clusters the sequences represented in each of the data sets, i.e. the sequences are clustered by transcript across datasets. For instance the same transcript may be represented in two or more datasets, where the sequence information for the transcript from each data set is clustered into one cluster, i.e. the information is pooled and clustered by transcript. In some embodiments, database manager 350 also eliminates redundant entries using the clustering method. For example manager 350 may employ a variety of clustering methods such as the well known BLAST method, or more preferably the BLAT method described in the following reference: Kent, W. J (2002) BLAT-the BLAST-like alignment tool; Genome Res., 12, 656-664; which is hereby incorporated by reference herein in its entirety for all purposes. In the present example, if the alignment of transcripts in a cluster overlap by >97% over their entire length, then manager 350 determines them to be redundant keeping the longest sequence and removing the one or more other sequences from the cluster. Also in the present example, preference may also be given to one or more transcripts from a particular data set, such as a dataset from RefSeq 307, where either the preferred sequence and/or the longest sequence is taken as representative of the cluster. In some implementations manager 350 stores the transcript clusters in transcript database 365 for use in one or more other methods or purposes.

Subsequently, step 415 illustrates the step of aligning the gene sequence of the transcript associated with the clusters with the probe sequences associated with the individual probe of each probe set. As illustrated in decision element 417, if>=9 probe sequences align perfectly to the gene sequence, then manager 350 assigns the relationship between the probe set and the transcript represented by the cluster as the highest level grade in step 420, such as for instance Grade A.

Manager 350 then aligns the representative sequence from each cluster with the gene sequence for the probe sets and transcripts that do not meet the criteria of decision element 417, as illustrated in step 425. If the alignment of the representative sequence and gene sequence overlaps with a target region as illustrated in decision element 427 of the sequence that represent the region interrogated by the probes of the probe set then manager 350 assigns the relationship between the probe set and the transcript represented by the cluster as the second highest level grade, such as for instance Grade B in step 430. For the remaining probe sets and transcript clusters that do not satisfy that criteria, if the alignment of the representative sequence and gene sequence overlap with each other, but do not overlap in the target region as illustrated in decision element 437 then manager 350 assigns the relationship between the probe set and the transcript represented by the cluster as the third highest level grade in step 440, such as for instance Grade C.

For the remaining probe sets and clusters, manager 350 may employ one or more mapping tools, such as for instance the BLASTX tool, to map the remaining clusters to the transcripts represented in a dataset from a preferred database as illustrated in step 445. For example, a dataset may be employed from UniGene 309 for the mapping of the remaining clusters, where if a cluster maps to a transcript represented in the preferred dataset according to decision element 447, then manager 350 assigns the relationship between the probe set that interrogates the transcript represented by the cluster as the fourth highest level grade in step 450, such as for instance Grade E. If not, manager 350 assigns the relationship between the probe set that interrogates the transcript represented by the cluster at the time of design as the fifth highest level grade in step 450, such as for instance Grade R.

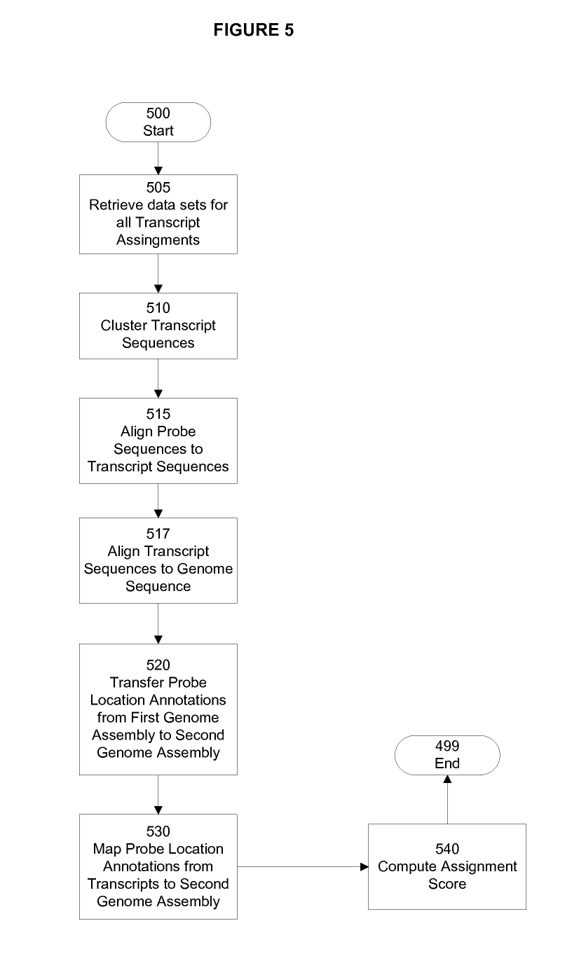

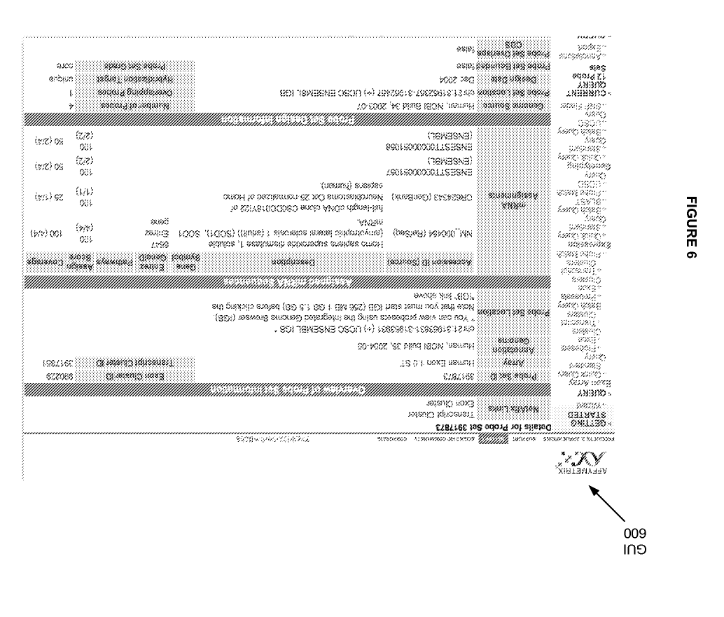

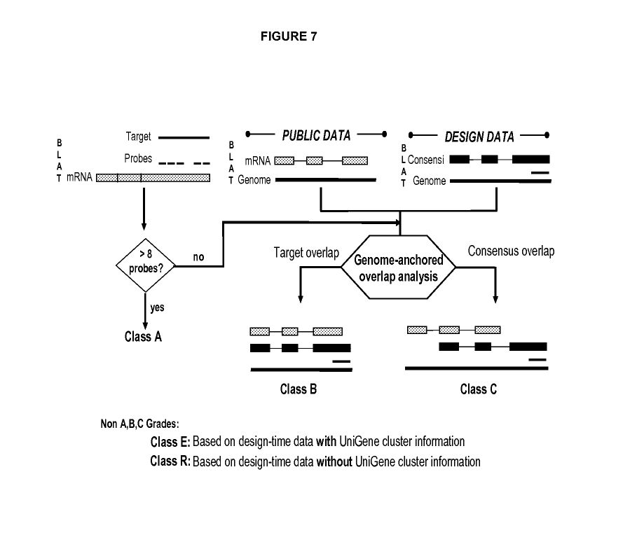

In the same or other embodiments one or more additional operations of updating annotation information may also be executed. FIG. 5 provides an illustrative example of one embodiment of a second method for updating annotation information associated with probe sets that interrogate the expression of one or more exons throughout a transcribed or putatively transcribed sequence that is representative of a gene of interest. Implementations of the presently described method may rely more heavily on alignments of probe and/or transcript sequences with genomic sequence that may be related to a more genome-based strategy for probe design. For example, the boundaries of an exon in a sequence may be known from experimental information, or alternatively the boundaries of an exon may be predicted using one or more computational or manual methods that identify signatures in a sequence indicative of an exon boundary. Such signatures could include identified internal splice sites, polyadenylation sites, CDS start and stop sites, or other types of signatures known to those of skill in the related art.

Steps 505 and 510 are similar to steps 405 and 410 described above with respect to FIG. 4, and in some embodiments steps 505 and 510 need not be performed because the clustered transcript data is available in transcript database 365.

Step 515 describes the operation of aligning probe sequence information to the clustered transcript information. For example, each transcript cluster has a representative sequence that may be employed to align associated probe sequences to. In the present example, more or fewer probe sequences may align to the representative sequence because there may be different sequence composition of the transcript sequence from the previous version.

In a step that may occur concurrently to step 515, the representative transcript sequences are also aligned to the most currently available genome sequence that is sometimes referred to as a “Build”. For example, the genomic sequence for a particular organism may be available form one or more resources that may include one or more databases 160 such as GenBank 305. In the present example, it is common that sequence builds contain some degree of error such as gaps in the sequence, erroneous repeats, misassembled sequence (i.e. region or sequence may be in an incorrect sequence position) or other type of possible error. Typically, sequencing efforts are on-going to resolve errors updating the corrected sequence in new builds that are released. Step 517, provides an example of aligning the transcript information to the most current sequence build.

Step 520 describes the step of transferring probe location information associated with a previous build to the most current build. For example, computational tools may be available to perform the operation of annotating the most current build with probe location information from the previous build such as what may be referred to as the liftOver tool developed by Jim Kent and available from the UCSC Genome Browser. In the present example, some probe location information may not transfer to the new build due to changes in sequence composition and arrangement associated with the new build. In some cases, the probe location information may be manually curated to the new sequence build, but it is also possible that a probe was designed to target a region of genome sequence that may not actually exist. In such cases, the erroneous probes may be identified and annotated as such or alternatively may be discarded.

Manager 350 may then transfer the probe locations aligned to the transcript to the genome sequence build as shown in step 530. Thus, the probes that are known to align with and target a transcript are mapped directly to the genome sequence. For example, the genome sequence build may be used as a template for direct comparison of probes that interrogate transcripts and all of the probes designed to interrogate a sequence region of the genome. In the present example, not all probes designed to target areas in a region of sequence associated with a transcript may actually target the transcript because of a number of possible factors including the exon composition and arrangement of the transcript.

Manager 350 may then calculate a quantitative score as described in step 540 using the number of actual probes that interrogate the transcript divided by the number of possible probes that could interrogate the transcript multiplied by 100. The score computed by manager 350 is representative of the degree of confidence that the probe set interrogates the assigned transcript, where a high score is associated with a high degree of confidence.

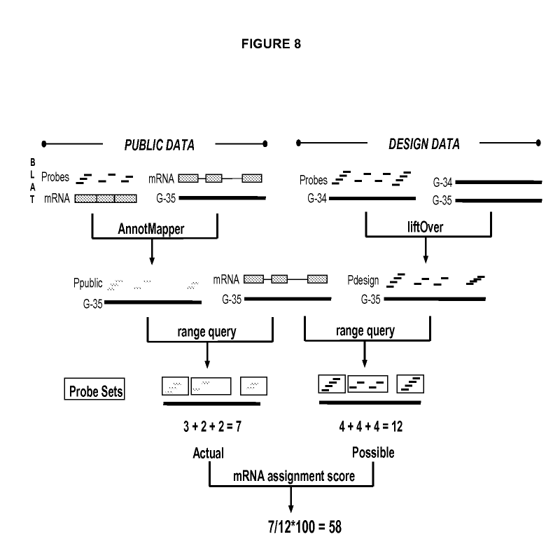

User 101 may, for example, obtain the updated annotation information described above in a variety of ways including selecting one or more probe set identifiers in one or more of GUI's 246 that may be received by input manager 345. Input manager 345 may forward the request from user 101 to database manager 350 which formulates one or more queries to interrogate annotation database 360. Database 360 returns annotation data to database manager 350 in response to the queries that is forwarded to output manager 347. Output manager 347 may then present the retrieved annotation information in one or more GUI's 246 that could be the same or different GUI that receives the probe set identifiers. In addition, output manager 347 may organize the annotation data into a plurality of GUI's 246 or pages that could be arranged in a hierarchical manner. For instance, the GUI's or pages may be organized so that annotation information for the transcript level may be at or near the top level. User 101 may navigate to lower levels using methods commonly used in the art such as selecting or clicking on or more graphical icons. In the present example, the lower levels could include an exon level, a probe set level, a probe level, or other levels that may provide a useful representation of annotation information to the user. GUI 600 of FIG. 6 provides an illustrative example of one such type of GUI.

FIG. 6 shows a capture of a Webpage for the NetAffx™ Analysis Center and it presents exon array data in hierarchical pages. One can drill down from the transcript cluster level down to the exon level or the probe set level. At any level, one can view information about assigned public mRNAs. Both quick and standard query interfaces are provided, similar to other Affymetrix arrays. Data can be exported into ExACT compatible format. Batch upload of probe set ids is available

The following discussion relates to a particularly preferred embodiment, one embodiment that can provide a computer system which is a dynamic, living catalog of gene sequence information and annotation for use with microarray probe information. mRNA and EST data is constantly being updated, evolving, and changing. So, incorporation of that data into one of the preferred embodiments, NetAffx, provides continuous advantages to a researcher who accesses the information over a network, like the internet, for example.

What is described is a computational pipeline for assigning public-domain mRNA sequences to probe sets on particularly preferred arrays, such as the Affymetrix GeneChip® Exon Array (commercially available from Affymetrix, Santa Clara). See also the following published applications which are hereby incorporated by reference in their entireties, publication number US 20050214823 A1 (Mouse sequence array) and publication number US 20050244851 A1 (Human sequence array), and include a comparison with the pipeline used for the Affymetrix expression arrays, some of which select probes at the 3′ end (see expression arrays listed above). Transcript assignment is a key step in the probe set annotation pipeline used by a preferred embodiment, the NetAffx™ Analysis Center, which has undergone enhancements since described in Liu et al. 2003, NAR 31: 82-86. See NetAffx at http://www.affymetrix.com/support/technical/whitepapers/Transcript Assignment whitepape r.pdf.

GeneChip® Exon Arrays permit exon-level expression profiling at the whole-genome scale and employ a design that is different from 3′ biased arrays (3′-arrays). The new transcript assignment pipelines for both exon arrays and 3′-arrays take advantage of whole genome sequence data but make use of it in different ways. We compare the design of the assignment pipelines for these different types of arrays with attention to differences in scoring strategy for ranking transcript assignments and for identifying potential cross-hybridization.

The record of publicly contributed mRNA sequences is continually evolving as new data are contributed to the public databases. The NetAffx mRNA database is updated periodically with public mRNA sequences from a variety of sources, such as Genbank, RefSeq, Ensembl, and other sources. This mRNA database is used for annotating all arrays having probe sequences directed to these sequences. To reduce redundancy, public mRNAs from GenBank and RefSeq are clustered together. As a reference data set, all RefSeq mRNAs are retained (even if there are multiple ones in a given cluster). For clusters containing only GenBank sequences, the longest sequence is selected as the cluster representative. Cross references to other databases are recovered using associated UniGene records. Ensembl mRNAs include known transcripts, EST-based transcripts, and predicted transcripts.

The challenges of maintaining this evolving sequence and annotation database include immature and fragmented mRNA records, inherited sequence errors from data used to design the chips and errors in mRNA and EST sequences. The NetAffx transcript assignment pipeline employs state of the art bioinformatics and assures the highest quality data possible.

To understand the methods described herein, it is useful to lay out some microarray probe design information, some of the preferred embodiments relating to GeneChip® expression arrays. See U.S. Pat. No. 6,826,296 which is hereby incorporated by reference in its entirety.

A typical probe set design is illustrated in FIG. 7. Typically, evidence of transcription (exemplar sequences) for a gene is exclusively EST data and this can happen when sequencing projects are new for an organism. The exemplars are clustered together by 30 similarity, combined by a clustering algorithm and abstracted into a single consensus sequence. From a subsequence of the consensus, the target sequence is extracted from which probes of the probeset are selected.

ESTs may have lower sequence accuracy, and if so, then the probe sequences can show a difference when compared to the genome sequence and transcript record over time.

The transcript record, mRNA and EST evidence, is constantly changing in databases around the world. mRNA sequences which may appear later, are usually truncated in the 5′ and 3′ ends so that EST based consensuses may not overlap consistently with subsequent mRNA sequences which they correspond to in vivo (top of FIG. 7). This can lead to omissions in transcript assignment when probe set matching to mRNA is used exclusively.

One preferred embodiment, NetAffx, employs a tiered assignment protocol. The NetAffx transcript assignment pipeline delivers the broadest assignment of mRNA transcript to probeset matching with the best reliability available.

FIG. 7 shows an overview of the pipeline to assign a public mRNA identification to probe sets on 3′ arrays and compute an assignment grade, a measure of the confidence that the probe set interrogates the assigned mRNA in microarray experiments. After consolidating the public mRNAs, a set of genomic alignments are created using the BLAT program (probe to mRNA, mRNA to genome, target to genome, and consensi to genome). BLAT is publicly available on the UCSC website. See http://genome.ucsc.edu/cgi-bin/hgBlat?command=start, see also See Kent, W. J (2002) BLAT-the BLAST-like alignment tool. Genome Res., 12, 656-664.

Genomic alignments are used secondarily, to differentiate the lower-grade assignments (B, C). Class A assignment is given if preferably more than 8 probes in the probe set perfectly match a given public mRNA. Higher cutoffs (11, 12, or 13) may be used with larger probesets. (Fewer probes, such as 7, 6, or 5 may also be used in other embodiments). This includes most probe sets on the more popular arrays (human, mouse, rat). All non class A assignments are assigned a B, C, E, or R grade. Class B assignment is given if an mRNA overlaps the probe set's target sequence in a genomic alignment by at least one base. Class C assignment is given if an mRNA overlaps the probe set's consensus sequence by at least one base. Class E and R are given to probes that fail the criteria for A, B, and C and derive from design-time mRNA data from UniGene. Class R assignments may represent a smaller fraction for arrays that have significant content from well studied organisms (human, mouse, rat) or a large fraction for arrays from less well studied organisms. As time progresses, more mRNAs are entered into Genbank and RefSeq, so the fraction of Class R assignments diminishes.

A, B, and C Grade probe sets have a transcript matched to them, and can be called Annotated probe sets. E and R Grade probe sets do not have a transcript matched to them and can be called unannotated or EST only probe sets. Use of Tiered Annotation Methods which are graded in their assignment content will allow the maximal extent to which the probe sets are Annotated and can result in a significant improvement in the documentation of the micro array as the transcript matures.

Instead of selecting probes only at the 3′ end of a single consensus or exemplar sequence, the exon array design process permits probes to be selected throughout multiple regions of transcribed or putatively transcribed sequence. Public domain input annotations are projected onto the genome to infer transcribed regions. Internal splice sites, polyadenylation sites, and CDS (coding sequence) start/stop positions are typically used to infer “hard edges” (the end of the sequence that defines the boundary of a PSR and cannot be extended beyond the border by other evidence) which resulted in the fragmentation of a contiguous piece of transcribed sequence (exon cluster) into multiple probe selection regions (PSRs).

FIG. 8 shows that the exon array public mRNA assignment pipeline employs the same consolidated public mRNAs, but the assignment scoring statistic is different here from the 3′-array approach. The exon array pipeline makes use of genome alignments, stemming from the highly genome-based strategy of the chip design. Probes are aligned to the public mRNAs and mRNAs are aligned to the genome (top left of diagram). The exon array annotation pipeline incorporates the genomic locations of the probes (top right of diagram) and uses sequence coordinate mapping tools. The liftOver tool (Jim Kent, UCSC, publicly available from the UCSC website) allows probe locations on one version of the genome assembly (eg. NCBI build 34) to be mapped into a newer version of the assembly (eg. NCBI build 35). Another software tool called AnnotMapper allows probe locations on an mRNA sequence to be mapped to locations in a genomic sequence, given the alignment of the mRNA to the genomic sequence. It can interconvert the gene boundary locations between the genomic coordinates, gene nucleotide coordinates (in the mRNA in processed or proto forms), and the polypeptide (protein product) coordinates. It is also used to map other related mRNA sequences to the genomic coordinates through the coordinates of the current mRNA. Converting the probe-to-mRNA alignment into a probe-to-genome alignment provides an efficient way to compare the number of probes which directly aligned to the mRNA (the “actual” number) to the number of probes which could potentially align to the mRNA (the “possible” number) based on the design-time probe genomic locations. Range queries are used to compare all probes alignments against all mRNA alignment blocks. To facilitate these range queries, an algorithm was designed that exploits information from genomic alignments to reduce the number of possible comparisons. An assignment score is computed for each probe set, calculated as actual probes/possible probes*100. Assignment scores for exon cluster and transcript cluster are derived by summing across probe sets with same exon cluster or transcript cluster ID. Note that the vast majority of probe sets contain four probes (1.2 M/1.4 M, 86%).

The scoring strategy for exon arrays provides a way to compare the potentially large number of different mRNA species which could be assigned to an array sequence, such as a transcript cluster which may contain hundreds of probe sets and span multiple megabytes of genomic sequence. The assignment score reflects the confidence that a given array sequence is interrogating the assigned mRNA (actual probes/possible probes multiplied by 100). A coverage score is also provided, which is proportional to the degree of overlap between the array sequence and the assigned mRNA (possible probes/total probes multiplied by 100). Smaller mRNA variants could have low coverage score yet still have a high assignment score. Note that for the exon arrays, antisense hits are not considered during design-time sequence selection and are currently not scored. Relationships between probesets and transcripts where fewer probe matches than the cutoff of 50% of the possible are kept as documentation of possible Cross hybridizing transcript records.

The pipeline typically associates nearly all of the sequence database with a probe set on the exon array. In one example 96.4% of all transcripts known are associated with an exon array probe set.

The raw assignment scores for transcript clusters on chromosome 21 are assigned to public mRNAs in the consolidated NetAffx mRNA database as of initial public launch of the Human Exon Array (27 Sep. 2005). The vast majority of scores (98.5%) are at the theoretical maximum of 100. A small fraction (1.4%) of scores exceed 100 due to overlap between near-identical mRNAs in their genomic alignment which escaped the clustering threshold used in the assignment pipeline.

### EXAMPLE

The Following is a Non-limiting Example of One Version of the Preferred Embodiment.

Non-redundant transcript database. mRNA sequences are obtained from the appropriate public databases (GenBank, RefSeq, Ensembl, Saccharomyces Genome Database, TIGR etc). The mRNA sequences for each organism are clustered at a high sequence identity (preferably over 85%, 90%, or 95%) and high alignment coverage (preferably over 85%, 90%, or 95%) using BLAT using sequence identity and alignment coverage as quality measures. See Kent, W. J (2002) BLAT-the BLAST-like alignment tool. Genome Res., 12, 656-664.

The longest sequence in each cluster is then used as the representative of that cluster, with preference given to RefSeq sequences. This non-redundant data set is the nucleotide record used for all transcript assignments for NetAffx. The peptide translation record for each transcript is also kept for protein annotation. E.g. GenBank release 142 has 135,632 mRNAs for Homo sapiens. Clustering at 90% sequence identity produced 61,950 clusters.

Probe matched transcript assignment. Pairwise alignment of the probe sequences with gene transcripts is the most accurate method to precisely determine the transcript sequences detected by probesets. See Chalifa-Caspi et al (2004) GeneAnnot: comprehensive two-way linking between oligonucleotide array probesets and GeneCards genes. Bioinformatics. 9, 1457-1458. All 25-mer probe sequences are aligned with the non-redundant mRNA set. mRNA sequences that match perfectly with at least 9 probes in a probeset are identified. These are referred to as “Matching Probe” or “Class A” assignments and represent the best quality assignments.

There are other relationships between a probe sequence and a transcript which may result. If an mRNA sequence is found to match less than a cutoff majority number (e.g. 9 out of 11 probes) perfectly in a probeset, it is recorded as a “cross-hyb” probeset. If the proper orientation of the consensus sequence constructed from mRNA and/or EST data is unknown at the time of design, probes are tiled against both the strands of the consensus sequence to ensure that the true transcript is represented on the array. If enough probes align with the negative strand of the mRNA then the corresponding probeset is annotated as “negative strand” probeset.

For example, consider probeset 200018_at from Affymetrix Human U133 Plus array. This probeset has 11/11 matching probes for BC00672 and 1 cross-hyb probe against X04297.

Probeset 1552279_a_at from Human U133 Plus array has 10/11 probes matching the sense strand of transcript AL832613 and 4/11 “negative strand” probes matching the anti-sense strand of RefSeq NM_{—}015077.

Genome based transcript assignment. If there are no adequate “Matching Probe” assignments for a probeset, then genomic alignments of the consensus/exemplar sequence are used. The consensus/exemplar sequences and the non-redundant mRNA sequences for each organism are aligned with the genomic sequence.

“Genome Target Overlap” (Class B) assignment are also based on genomic alignments where the target region aligns with the genome and only partially overlaps with the mRNA->genome alignment. These could be the result of an incomplete EST-based extension of the 3′ region of the transcript.

If the target region of the consensus/exemplar sequence aligns with the genome and does not overlap with the genomic alignment of an mRNA, then the transcript assignment is annotated as “Genome Consensus/Exemplar Overlap” (Class C). These alignments must meet identity and alignment coverage cutoffs. Therefore, even if one nucleotide of the target sequence overlaps with the mRNA alignment it is recorded as “Consensus/Exemplar Overlap”. Several mRNA sequences with incomplete 3′ UTR sequences may not overlap significantly with the 5′ of a consensus sequence based on the EST data. But, if their placement can be verified by placement on the genome, then the assignment has some significant evidence.

Exemplary benefits of the present invention show that as an organism's genome and transcript record become more mature, the EST only (E and R) probe sets are being converted to A B and C assignments. Also, using a tiered combination of A, B, and C adds a considerable amount of annotation coverage to the arrays—7-16% in these three cases, again more helpful with newer arrays and organism efforts.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

Also, the functions of several elements may, in alternative embodiments, be carried out by fewer, or a single, element. Similarly, in some embodiments, any functional element may perform fewer, or different, operations than those described with respect to the illustrated embodiment. Also, functional elements shown as distinct for purposes of illustration may be incorporated within other functional elements in a particular implementation. Also, the sequencing of functions or portions of functions generally may be altered. Certain functional elements, files, data structures, and so on may be described in the illustrated embodiments as located in system memory of a particular computer. In other embodiments, however, they may be located on, or distributed across, computer systems or other platforms that are co-located and/or remote from each other. For example, any one or more of data files or data structures described as co-located on and “local” to a server or other computer may be located in a computer system or systems remote from the server. In addition, it will be understood by those skilled in the relevant art that control and data flows between and among functional elements and various data structures may vary in many ways from the control and data flows described above or in documents incorporated by reference herein. More particularly, intermediary functional elements may direct control or data flows, and the functions of various elements may be combined, divided, or otherwise rearranged to allow parallel processing or for other reasons. Also, intermediate data structures or files may be used and combined or otherwise arranged. Numerous are contemplated as falling within the scope of aims and equivalents thereto.

1. A method of assigning transcripts to probe sets, comprising:
retrieving one or more data sets from one or more sources;
generating one or more clusters using transcript data from the data sets;
aligning one or more probe sequences to a representative sequence from one or more of the clusters;
aligning the representative sequence to a genome sequence, wherein the genome sequence is annotated with probe location information;
mapping the aligned probe sequences to the genome sequence using the alignment of the representative sequence and genome sequence;
computing a score using a number associated with the aligned probe sequences and a number associated with the probe location formation associated with a region of the genome sequence that corresponds to the aligned representative sequence.
